(19) United States
(12) Patent Application Publication
(10) Pub. No.: US 2016/0058549 A1
(43) Pub. Date: Mar. 3, 2016

(54) LIGAMENT FIXTURE

(71) Applicant: TAKIRON CO., LTD., Osaka (JP)

(72) Inventors: Minoru NODA, Osaka (JP); Hiroshi MORII, Osaka (JP)

(73) Assignee: TAKIRON CO., LTD., Osaka (JP)

(21) Appl. No.: 14/837,627

(22) Filed: Aug. 27, 2015

(30) Foreign Application Priority Data
Aug. 29, 2014 (JP) 2014-175461

(51) Int. Cl. A61F 2/08

(52) U.S. Cl. CPC A61F 2/0811; A61F 2002/0888; A61F 2210/0004; A61F 2002/0852; A61F 2002/0817

(57) ABSTRACT

A ligament fixture to be inserted and fixed into a fitting hole formed in an in-vivo bone is provided, and includes an in-vivo degradable and absorbable material which is a polymer-bioceramic composite material. The ligament fixture has at least one pair of opposed surfaces which are inclined faces gradually getting closer to each other toward to a front-end side of the ligament fixture and which form a wedge shape having a resistance imparting parts on each of the inclined faces. The ligament fixture includes a front-end part having a cross-sectional profile smaller than a cross-sectional profile of the fitting hole, and a main body part continuing from the front-end part, the main body part including a section having a cross-sectional profile larger than the cross-sectional profile of the fitting hole.

### CROSS-REFERENCE TO RELATED APPLICATION

This application claims the benefit of Japanese Patent Application No. 2014-175461, filed on Aug. 29, 2014, the entire contents of which are hereby incorporated by reference.

### BACKGROUND OF THE INVENTION

1. Technical Field

The present invention relates to a ligament fixture to be inserted and fixed into a fitting hole formed in an in-vivo bone.

2. Background Art

In an existing reconstructive surgery of an anterior cruciate ligament (ACL) torn, a ligament or tendon (hereafter collectively referred to as “ligament”) picked out of another part of the human body are implanted into the knee joint concerned.

And in such ligament implantation, as shown e.g. in FIG. 5A, holes H1 and H2 are drilled through in-vivo bones (articular bones) B1 and B2, respectively, which are situated on opposite sides of the fitting place of a ligament M, and by the use of a string-shaped connecting member W2 as a guiding suture, which is connected to the ligament M through the medium of a bone piece Bo, the ligament M, together with the string-shaped connecting members W1 and W2, each of which is connected to the ligament M via its own bone piece Bo, is pulled into the holes so as to head toward the hole H2 formed in the in-vivo bone B2 from the hole H1 formed in the in-vivo bone B1, and so that the connecting members W1 and W2 are situated in the holes H1 and H2 formed in the in-vivo bones B1 and B2, respectively, and the ligament M is situated between the connecting members.

Further, at the end of connecting member W2 drawn out from a mouth of the hole H2 formed in the in-vivo bone B2 is arranged a ligament fixture B having the shape of a button and a size large enough to lock the mouth of the hole H2, and through the ligament fixture B, the end of the connecting member W2 is engaged in the mouth of the hole H2. In this situation, an interference screw S including an in-vivo degradable and absorbable material is screwed into the hole H1 formed in the in-vivo bone B1, and thereby the bone piece Bo is engaged so as to be put between the circumferential wall of the hole H1 and the peripheral face of the interference screw. In this way, the implantation of the ligament M is completed.

Alternatively, the engagement of the connecting member W1 in the hole H1 may be carried out, as in the case of the connecting member W2, by the use of a ligament fixture having the shape of a button and a size large enough to lock the mouth of the hole H1.

Incidentally, an existing method for implanting a ligament requires that the operation for engaging the end of the connecting member W1 in the mouth of the hole H1 via the button-shaped ligament fixture B should be performed in the interior of the human body, and hence has problems with its certainty and operability.

In addition, the button-shaped ligament fixture B is in a condition that the ligament fixture B protrudes from the surface of the in-vivo bone B2, and sometimes it has produced detrimental effects including inflammation during the convalescence.

Moreover, in order to address such a problem, it becomes necessary to form, as shown in FIG. 5B, at the mouth of the hole H2 formed in the in-vivo bone B2 a countersunk hole H2b into which the button-shaped ligament fixture B fits, but this approach is impractical because it puts a significant burden on patients.

Additionally, the traverse-sectional profile of a ligament M to be implanted is generally long in a lateral direction (rectangular or elliptic in shape), and in order to adapt to such a shape it becomes necessary for the hole H1 formed in the in-vivo bone B1, into which the ligament M is to be pulled, to be large in its inside diameter circumscribing the ligament M, which situation also causes a problem that a significant burden puts on patients.

### SUMMARY

In view of the problems concerning an existing method for implanting a ligament, notably the ligament fixture used therein, an object of the invention is to provide a ligament fixture which ensures high certainty and operability of fixation of connecting members connected to a ligament, and besides which allows not only easy adaptation to ligaments having a traverse-sectional profile long in their lateral directions but also reduction in burdens on patients.

According to an aspect of the invention, there is provided a ligament fixture to be inserted and fixed into a fitting hole formed in an in-vivo bone, comprising an in-vivo degradable and absorbable material which is a polymer-bioceramic composite material. The ligament fixture has at least one pair of opposed surfaces which are inclined faces gradually getting closer to each other toward to a front-end side of the ligament fixture and which form a wedge shape having a resistance imparting parts on each of the inclined faces. The ligament fixture comprises a front-end part having a cross-sectional profile smaller than a cross-sectional profile of the fitting hole, and a main body part continuing from the front-end part, the main body part comprising a section having a cross-sectional profile larger than the cross-sectional profile of the fitting hole.

In this aspect, the other pair of opposite surfaces may be configured to be inclined faces gradually getting closer to each other toward the front-end side of the ligament fixture.

The resistance imparting parts may be provided on the section having the cross-sectional profile larger than the cross-sectional profile of the fitting hole.

The resistance imparting parts may be formed so as to protrude from the inclined faces.

The resistance imparting parts may be formed with recesses provided in the inclined faces.

A linkage site for connecting an operation tool may be provided in a rear-end part of the ligament fixture.

In an intermediate part of the ligament fixture, a through hole for engaging therein a connecting member to be connected to a ligament may be formed, and a groove may be formed from the through hole toward the front-end part for placing the connecting member.

Further, a communicating hole ranging from a rear-end face of the ligament fixture to the through hole in the intermediate part of the ligament fixture may be formed, and an intermediate part of the connecting member to be connected to the ligament may be positioned in a form of a loop at an opening part of the communicating hole on a rear-end face of the ligament fixture, and both end parts of the connecting member may be disposed so as to be pulled out of both openings of the through hole, respectively.

The ligament fixture may be made from the composite material having undergone reinforced molding.

The ligament fixture may be configured to be inserted and fixed into the fitting hole formed in an in-vivo bone in a condition of being wholly buried in the fitting hole.

The ligament fixture may be configured to be inserted and fixed into the fitting hole formed in an in-vivo bone by means of a bone hole forming tool so as to have a cross-sectional profile invariant in a depth direction.

The ligament fixture may be configured to be inserted and fixed into a fitting hole formed in an in-vivo bone by means of a bone hole forming tool so as to have a cross-sectional profile gradually reducing in a depth direction.

According to an aspect of the invention, the ligament fixture includes an in-vivo degradable and absorbable material which is a polymer-bioceramic composite material. The ligament fixture having at least one pair of opposed surfaces which are inclined faces gradually getting closer to each other toward to a front-end side of the ligament fixture and which form a wedge shape having a resistance imparting parts on each of the inclined faces. The ligament fixture comprises a front-end part having a cross-sectional profile smaller than a cross-sectional profile of the fitting hole, and a main body part continuing from the front-end part, the main body part comprising a section having a cross-sectional profile larger than the cross-sectional profile of the fitting hole. Thus, by virtue of its wedge action, the ligament fixture can be inserted and fixed into a fitting hole formed in an in-vivo bone with certainty in a stabilized condition and allows fixation of the connecting member connected to a ligament with high certainty and operability, and not only can easily adapt to a ligament having a traverse-sectional profile long in its lateral direction but also, in combination with its composition including an in-vivo degradable and absorbable material, allows reduction in burdens on patients, inclusive of those during the convalescence, by making the shape of a fitting hole formed in an in-vivo bone have a minimum size required to adapt the hole to the shape of the ligament.

In addition, the other pair of opposed surfaces thereof may be configured to be inclined faces gradually getting closer to each other toward the front-end side. Thus, the ligament fixture can be fixed more certainly into a fitting hole formed in an in-vivo bone by virtue of wedge action of its four surfaces.

Further, the resistance imparting parts may be provided on the section having the cross-sectional profile larger than the cross-sectional profile of the fitting hole. Thus, the ligament fixture can be inserted and fixed into a fitting hole formed in an in-vivo bone with certainty in a more stabilized condition.

Furthermore, the resistance imparting parts may be formed so as to protrude from its inclined faces or by providing recesses in its inclined faces. Thus, the ligament fixture can be fixed into a fitting hole formed in an in-vivo bone with higher certainty through the wedge action of its faces having such resistance imparting parts.

In addition, a linkage site for connecting an operation tool may be provided in a rear-end part of the ligament fixture. Thus, the ligament fixture can be inserted and fixed with certainty into a fitting hole formed in an in-vivo bone by receiving a blow or the like thereon through the medium of the operation tool.

Further, in an intermediate part of the ligament fixture, a through hole for engaging therein a connecting member to be connected to a ligament may be formed, and a groove may be formed from the through hole toward the front-end part for placing the connecting member. Thus, the ligament fixture can be inserted and fixed into a fitting hole formed in an in-vivo bone in a state of being connected to the connecting member without causing interference with the fitting hole formed in the in-vivo bone.

Furthermore, a communicating hole ranging from a rear-end face of the ligament fixture to the through hole in the intermediate part of the ligament fixture may be formed, and an intermediate part of the connecting member to be connected to the ligament may be positioned in a form of a loop at an opening part of the communicating hole on a rear-end face of the ligament fixture, and both end parts of the connecting member may be disposed so as to be pulled out of both openings of the through hole, respectively. Thus, the ligament fixture and a ligament can be connected easily and certainly with the connecting member

In addition, the ligament fixture may be made from the composite material having undergone reinforced molding. Thus, the ligament fixture can be prevented with certainty from suffering breakage or damage at the time when it is inserted and fixed into a fitting hole formed in an in-vivo bone by receiving a blow or the like thereon through the medium of the operation tool.

And, the ligament fixture may be configured to be inserted and fixed into the fitting hole formed in an in-vivo bone in a condition of being wholly buried in the fitting hole. Thus, it can be prevented from causing detrimental effects, inclusive of inflammation during the convalescence, by protrusion of the fixture through the surface of the in-vivo bone.

Moreover, by configuring the ligament fixture to be inserted and fixed into a fitting hole which is formed in an in-vivo bone by means of a bone hole forming tool so that the cross-sectional profile of the hole is invariant in a depth direction, or by configuring the ligament fixture to be inserted and fixed into a fitting hole which is formed in an in-vivo bone by means of a bone hole forming tool so that the cross-sectional profile of the hole is gradually reduced in a depth direction, insertion and fixation conditions of the ligament fixture can be adjusted for the shape of the fitting hole with reference to the properties of the in-vivo bone targeted for application.

### BRIEF DESCRIPTION OF THE DRAWINGS

FIGS. 1A and 1B show an embodiment of a ligament fixture according to an aspect of the invention, where FIG. 1A is a plan view and FIG. 1B is a side view.

FIGS. 2A and 2B show a modified embodiment of a ligament fixture according to an aspect of the invention, where FIG. 2A is a plan view and FIG. 2B is a side view.

FIGS. 3A-3F show an embodiment of a bone hole forming tool, where FIG. 3A is a front view, FIG. 3B is a plan view, FIG. 3C is a plan view of the front end, FIG. 3D is a side view of the front end, FIG. 3E is a side view of the base end and FIG. 3F is an explanatory drawing of the lower hole.

FIGS. 4A and 4B are explanatory drawings of a method for implanting a ligament through the use of a ligament fixture according to an aspect of the invention.

FIGS. 5A and 5B are explanatory drawings of an existing method for implanting a ligament.

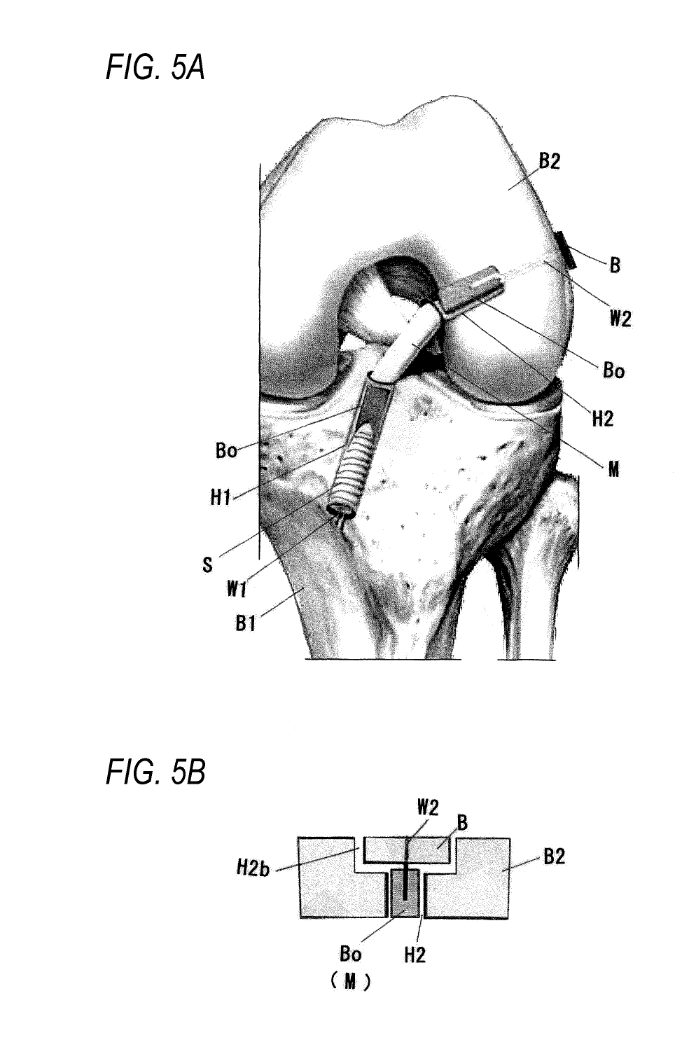

FIGS. 6A-6C show another modified embodiment of a ligament fixture according to an aspect of the invention, where FIG. 6A is a plan view, FIG. 6B is a side view and FIG. 6C is a profile appearing by cutting at the X-X line in FIG. 6A.

FIGS. 7A-7D are explanatory drawings showing a process for, by the use of the ligament fixture, connecting the ligament fixture and a piece of bone communicated to a ligament with a connecting member.

FIGS. 8A and 8B show still another modified embodiments of a ligament fixture according to an aspect of the invention, where FIG. 8A is a plan view and FIG. 8B is a side view.

### DETAILED DESCRIPTION OF THE INVENTION

In the following, embodiments of a ligament fixture according to an aspect of the invention are illustrated on the basis of drawings.

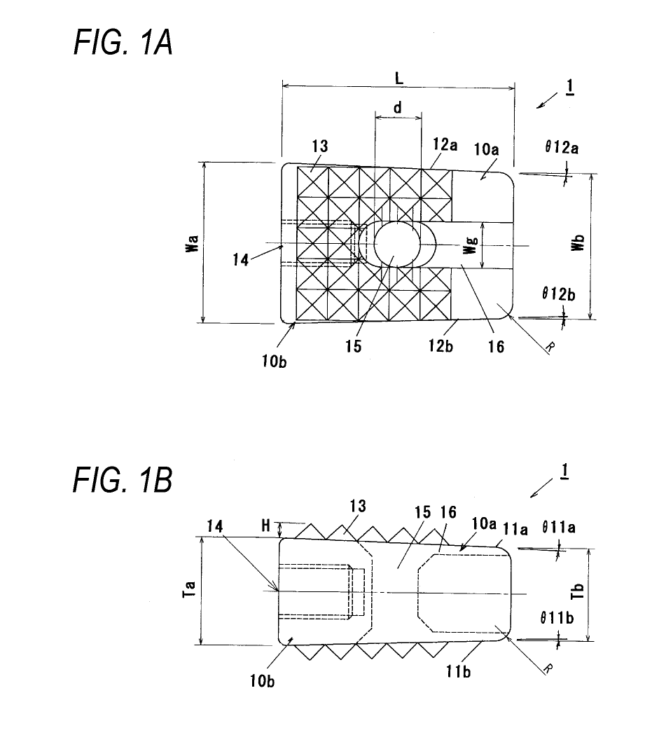

An embodiment of a ligament fixture according to an aspect of the invention is shown in FIG. 1.

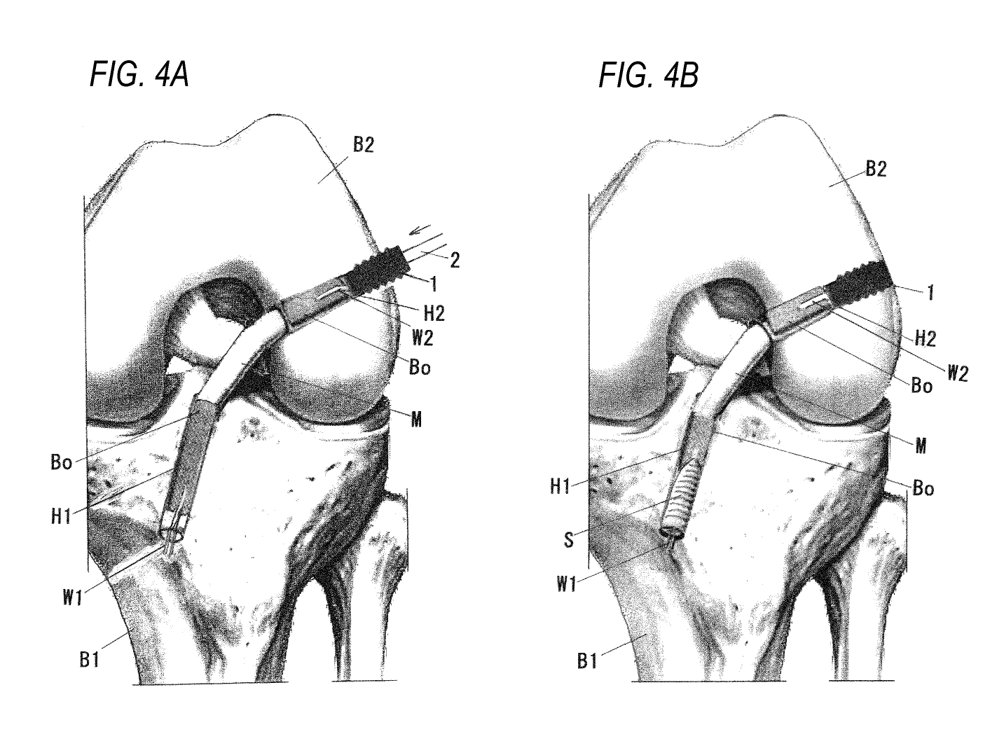

This ligament fixture 1 is, as shown in FIGS. 4A and 4B, used in a state of being inserted and fixed into a fitting hole H2 formed in an in-vivo bone B2. The ligament fixture 1 includes an in-vivo degradable and absorbable material, specifically a polymer-bioceramic composite material, and is configured to have at least one pair of opposed surfaces 11a and 11b which are inclined faces gradually getting closer to each other toward the front-end side and which form a wedge shape having a resistance imparting part 13 on each of the inclined faces, in which the front-end part 10a have a cross-sectional profile smaller than the cross-sectional profile of the fitting hole H2 and the main body part 10b continuing from the front-end part 10a have a section formed so that the cross-sectional profile is larger than the cross-sectional profile of the fitting hole H2.

Thus, by virtue of its wedge action, the ligament fixture 1 can be inserted and fixed into the fitting hole H2 formed in the in-vivo bone B2 with certainty in a stabilized condition, ensures high certainty and operability in fixing a string-shaped connecting member W2 as a suture, which is connected to a ligament M, and not only can easily adapt to the ligament M having a transverse-sectional profile long in its lateral direction but also, in combination with its composition including an in-vivo degradable and absorbable material, allows reduction in burdens on patients, inclusive of those during the convalescence, by making the shape of a fitting hole H2 formed in an in-vivo bone B2 have a minimum size required to adapt the hole H2 to the shape of the ligament M.

Further, in this embodiment of the ligament fixture 1, the other pair of opposed surfaces 12a and 12b are also configured to have inclined faces gradually getting closer to each other toward the front-end side.

Thereby, through the wedge action of the four faces thereof, the ligament fixture 1 can be fixed more certainly into the fitting hole formed in the in-vivo bone.

In this case, as to the angles of inclination of the center lines on the two opposed surfaces 11a and 11b which form the inclined faces gradually getting closer to each other toward the front-end side of the ligament fixture 1 and those on the other pair of opposed surfaces 12a and 12b with respect to their respective parallel planes, which angles are represented as θ11a, θ11b, θ12a and θ12b, respectively, it is preferred that such angles be chosen from a range of 0.5 to 10 degrees and adjusted to make not only the two opposed surfaces 11a and 11b but also the other pair of opposed surfaces 12a and 12b symmetric with respect to the center line. However, it is also possible to choose an angle of inclination from another angle range. For instance, one of two opposed faces may be made parallel to the center line (inclined angle θ: 0 degree), or two opposed surfaces may be made asymmetric by giving them different angles of inclination.

In addition, one of two pairs of opposed surfaces, specifically the other pair of opposed surfaces 12a and 12b, can be formed with flat faces as in this embodiment, or equivalently, the transverse-sectional profile of the ligament fixture 1 can be formed into the shape of a rectangle, but they may be formed with curved faces (semicircular faces), or equivalently, the transverse-sectional profile of the ligament fixture 1 may be formed into the shape of an ellipse.

Depending on the size of the fitting hole H2 formed in the in-vivo bone B2 and required to have a size suited to the shape of the ligament M to be fixed, the size of the ligament fixture 1 is adjusted e.g. as follows. Length L: on the order of 12 mm to 23 mm, Rear-end width Wa: on the order of 8 mm to 12 mm, Front-end width Wb: on the order of 7 mm to 11 mm, Rear-end thickness Ta: on the order of 5 mm to 8 mm, and Front-end thickness Tb: on the order of 4 mm to 7 mm.

Herein, in order that operations of putting the front-end part 10a of the ligament fixture 1 to the mouth of a fitting hole H2 formed in an in-vivo bone and inserting-and-fixing the fixture 1 into the fitting hole H2, which operations are required to be performed in a living body, are carried out with certainty in a stabilized condition, it is preferred that the edges of the front-end part be rounded (shaped into a curved face) on the order of R: 0.5 to 1.5 mm.

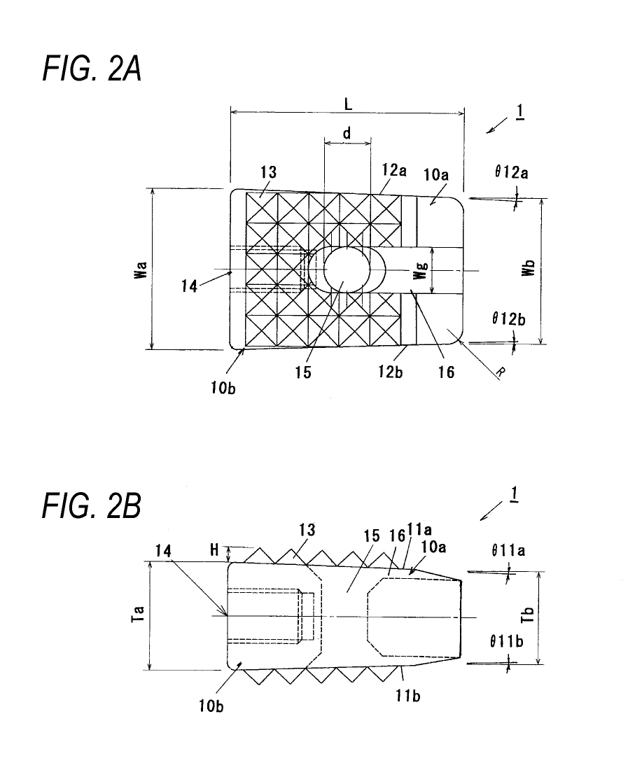

In addition, it is also preferred that the cross-sectional profile of the forward extremity of the front-end part 10a be shaped to become one size smaller than the cross-sectional profile of the fitting hole H2 formed in the in-vivo bone B2. As an example of a technique for realizing such a profile, mention may be made of such a technique that, like the modified embodiment of the ligament fixture 1 shown in FIGS. 2A and 2B, the angles of inclination θ11a and θ11b of the two opposed surfaces 11a and 11b are made greater than the angle of inclination of the main body part.

The resistance imparting part 13 is configured to be provided on a section where the cross-sectional profile of the main body part 10b is shaped to become larger than the cross-sectional profile of the fitting hole H2.

By doing so, the operations for fitting the ligament fixture 1 in the human body, and more specifically, operations of putting the ligament fixture 1 to the mouth of the fitting hole H2 formed in an in-vivo bone and inserting-and-fixing the fixture 1 into the hole H2, can be carried out with certainty in a stabilized condition.

The resistance imparting part 13 can be formed so as to protrude from each of one pair of opposed surfaces 11a and 11b formed with inclined faces, or it can be formed by providing recesses in the inclined faces (Illustration of this case is omitted).

And the shape of the resistance imparting part 13 in a protruded state can be formed with protrusions or projected streaks having any of shapes capable of imparting resistance. In this embodiment, protrusions having a high strength and a shape like a tetragonal pyramid (base: 1.5-2.5 mm×1.5-2.5 mm, height H: 0.5-1.5 mm) are preferably adopted.

Thus, by virtue of wedge action of surfaces at which the resistance imparting parts of the ligament fixture 1 are provided, it becomes possible to fix with more certainty the ligament fixture 1 in the fitting hole H2 formed in the in-vivo bone B2. More specifically, through the wedge function of sets of 4 faces formed on each resistance imparting part 13, the ligament fixture 1 can be fixed more certainly in the fitting hole formed in the in-vivo bone.

By the way, though no resistance imparting part 13 is formed on each of the other pair of opposed surfaces 12a and 12b in this embodiment, the resistance imparting part 13 can also be formed on each of the other pair of opposed surfaces when required.

The ligament fixture 1 is configured so that at its rear end is formed a linkage site 14 for connecting an operation tool 2 with which are performed operations for fitting the ligament fixture 1 in the human body, namely operations of putting the ligament fixture 1 to the mouth of the fitting hole H2 formed in an in-vivo bone from the outside of the human body and inserting-and-fixing the fixture 1 into the hole H2.

As to the operation tool 2 which can be used in this embodiment, its front-end side to be inserted into the human body is formed into the shape of a rod, and its base-end side situated outside the human body is made so as to function as an operation site to which a blow or the like can be delivered (Illustration of this tool is omitted).

And as the linkage site 14 can be adopted a structure capable of being easily attached to and detached from the tip of the operation tool 2, specifically a screw-threaded engagement structure using a female thread, a fit-and-bond structure, a clip structure or so on.

Thus the ligament fixture 1 can certainly be inserted and fixed into the fitting hole H2 formed in the in-vivo bone B2 by receiving a blow or the like through the medium of the operation tool 2.

The ligament fixture 1 is configured not only to have in its intermediate part a through hole 15 for engaging therein a connecting member W2 to be connected to the ligament M but also to form a groove 16 extending at least from the through hole 15 toward its front end in order to place therein the connecting member W2.

By doing so, in a state that the connecting member W2 is connected to the ligament fixture 1, the ligament fixture 1 can be inserted and fixed into the fitting hole H2 formed in the in-vivo bone B2 without causing interference with the fitting hole H2 formed in the in-vivo bone B2.

It is possible to form the ligament fixture 1 from a reinforced and molded polymer-ceramic composite material.

And thereby the ligament fixture 1 can certainly be prevented from suffering breakage or damage at the time when it is inserted and fixed into the fitting hole H2 formed in the in-vivo bone B2 by delivering a blow or the like thereto through the medium of the operation tool 2.

Here is given an explanation for an in-vivo degradable and absorbable material which is obtained by reinforcing and molding a polymer-bioceramic composite and used in forming the ligament fixture 1.

Such an in-vivo degradable and absorbable material is the material proposed before as an implant material by the present applicant (if necessary, see e.g. Japanese Patent No. 3239127), and includes a composite material containing a surface-bioactive bioceramic powder measuring 0.2 μm to 50 μm in particle size or aggregate size in an amount that the powder constitutes 10 wt % to 60 wt % of the composite material in a state of being dispersed homogeneously in a substantial sense into a basically in-vivo degradable and absorbable, crystalline thermoplastic polymer, wherein the polymer is crystallized and aligned through the pressurization by press-fit filling, and besides, the polymer has a crystallinity of 10% to 70%; or equivalently, a reinforced particles-matrix polymer composite material including a pressure-aligned compact obtained by compression molding or forging molding in which pressure alignment is performed by press-fit filling into a closed mold.

### (A) Materials

### (a) Bioceramics

### 1) Bioceramics

The bioceramics are surface-bioactive bioceramics.

Examples of surface-bioactive bioceramics include fired hydroxyapatite (HA), bioglass-base or crystallized glass-base biocompatible glass, diopside and mixtures of two or more of the above. Among them, preferably usable ones are fired hydroxyapatite (HA), bioglass-base biocompatible glass, cerabital, crystallized glass-base A-W glass ceramic, crystallized glass-base biopellet-1, implant-1, β-crystallized glass and diopside.

### 2) Other Bioceramics

As bioceramics other than the surface-bioactive bioceramics, bioceramics like in-vivo absorbable bioceramics can also be used.

Examples of in-vivo absorbable bioceramics include unfired hydroxyapatite, dicalcium phosphate, tricalcium phosphate, tetracalcium phosphate, octacalcium phosphate, calcite and mixtures of two or more of the above. Among them, preferably usable ones are unfired hydroxyapatite (unfired HA), dicalcium phosphate, α-tricalcium phosphate (α-TCP), β-tricalcium phosphate (β-TCP), tetracalcium phosphate (TeCP), octacalcium phosphate (OCP), dicalcium phosphate dihydrate-octacalcium phosphate (DCPD-OCP), dicalcium phosphate anhydride-tetracalcium phosphate (DCPA-TeCP), calcite and so on.

The bioceramics 1) differ from the bioceramics 2) in the degree of bioactivity, and this difference brings about differences in the speed of forming a new bone and the shape of a newly formed bone. Thus, in order to secure required bioactivity, some of those bioceramics may be used alone, or mixtures of two or more different bioceramics chosen as appropriate may be used. Accordingly, the expression of “surface-bioactive bioceramics” as used herein includes not only individual surface-bioactive bioceramics but also mixtures of surface-bioactive bioceramics as main ingredients and small amounts of in-vivo absorbable bioceramics. By the way, the unfired HA as an in-vivo absorbable bioceramic included in 2) differs from fired HA included in 1) but has a high degree of similarity to HA in a living body, and therefore it is absorbed and disappears thoroughly in a living body. And unfired HA is one of the most effective, in-vivo absorption-active powders because it is high in activity and safety and also has a proven track record in practical use.

### (b) Particle Size of Bioceramic Powder

The term “bioceramic powder” as used herein refers to the generic name for primary bioceramic particles and secondary bioceramic particles as aggregates (agglomerations).

Where the particle size thereof is concerned, the surface-bioactive bioceramic powder having its primary particle size or aggregate (agglomeration) size (secondary particle size) in a range of 0.2 μm to 50 μm, preferably 1 μm to 10 odd μm, is used in order to obtain a high-strength composite material on the basis of the foregoing reason. Also from the viewpoint of homogeneously dispersing the powder into an in-vivo degradable and absorbable, crystalline thermoplastic polymer, it is appropriate that the particle size of a surface-bioactive bioceramic powder be in the foregoing range. When the particle size of a surface-bioactive bioceramic powder is close to the upper limit of 50 μm, it is preferred that the powder be in the state of secondary particles as aggregates of primary particles around ten odd μm in size. On the other hand, it is undesirable for primary particles in isolation from one another to have sizes close to 50 μm because the resultant composite suffers breakage (rupture) at the time of yielding. The pressure aligned compact formed by press-fit filling can be finished off, if necessary, so as to have various precise shapes by machining or the like. However, when the powder is large in particle size, the compact is difficult to machine into a fine and precise shape because it becomes chipped or cracked at interfaces between particles. Thus it can be said that the particle size of 50 μm is the upper limit defining preciseness of the shape.

On the other hand, the particle size of 0.2 μm as the lower limit is comparable e.g. to the primary particle size of unfired HA. By the way, it has been ascertained that the fired HA used as surface-bioactive bioceramic in the invention has almost the same particle size as the primary particle size of unfired HA. In general, these fine particles aggregate and form secondary coagulated particles from several μm to ten odd μm in size. By obtaining a system in which particles or aggregates of a surface-bioactive bioceramic having their apparent average particle size in the foregoing range are dispersed homogeneously into a polymer matrix, the system satisfies the requirement for simultaneous possession of high strength and the property of being speedily replaced with an in-vivo bone through the absorption thereof. Thereby a composite material precise in shape can be obtained.

When a material containing such a surface-bioactive bioceramic is implanted into a living body, the surface-bioactive bioceramic powder having come to the material surface is bonded to an in-vivo bone surrounded by the material directly without the medium of fibrous connective tissue or indirectly through the medium of HA deposited on the surface, and thereby initial fixation is achieved at an early stage between the material and the in-vivo bone.

### (c) Polymer

The polymer has no particular restrictions so long as it is included in in-vivo degradable and absorbable, crystalline thermoplastic polymers, but preferred ones among them are polylactide and various lactide copolymers (e.g. lactide-glycolide copolymer) whose biological safety and biocompatibility have been verified and already been in practical use.

As the polylactide, homopolymer of L-lactide or D-lactide is suitable and, as the lactide-glycolide copolymer, one which has a molar ratio within a range of 99:1 to 75:25 is suitable because it has higher hydrolysis resistance than homopolymer of glycolide. Therein may be admixed a small amount of noncrystalline poly-D-lactide or poly-L-lactide, D- or L-lactide-glycolide copolymer, lactide-caprolactone copolymer or another in-vivo degradable and absorbable polymer compatible with the above-recited homopolymers or copolymers for the purpose of enhancing ease of plastic deformation or imparting tenacity to an aligned compact obtained by pressurized alignment. Considering the reaction with a living body or their degradation speeds, polymer products reduced in unreacted monomers and catalyst residues by removal and purification are preferred as a matter of course.

### (d) Raw Material Polymer and Molecular Weight of Preliminary Molded Body

Although the polymer as an osteosynthesis material is required to ensure a physical property such as strength of at least a certain value or higher, the molecular weight thereof is absolutely lowered at the stage of melt-molding the polymer into a preliminary molded body such as a billet, and it is therefore important to use the polymer having an initial viscosity-average molecular weight of from 150000 to 700000, and preferably from 250000 to 550000, when the polymer is polylactide or lactide-glycolide copolymer. When the polymer having its molecular weight in the forgoing range is used and subjected to melt molding under heating, it becomes possible to obtain finally a preliminary molded body having a viscosity average molecular weight of from 100,000 to 600,000. In other words, the in-vivo degradable and absorbable, crystalline thermoplastic polymer is polylactide or lactide-glycolide copolymer having an initial viscosity-average molecular weight of from 150,000 to 700,000, and it comes to have a viscosity-average molecular weight of from 100,000 to 600,000 after melt molding.

The polymer can be converted into a high-strength composite material by cold plastic deformation for molecular chain (crystal) alignment according to pressurized alignment by subsequent press-fit filling, and the lowering of the molecular weight can be minimized if this plastic deformation process is operated under appropriate condition settings.

When the initial viscosity-average molecular weight of the polymer is lower than 150000, there is an advantage in that molding is easy because the melt viscosity is low, but high initial strength cannot be obtained, and besides, because of fast strength drop in a living body, a strength retention period becomes shorter than a period required for bone healing. On the other hand, when the initial viscosity-average molecular weight of the polymer is excessively increased beyond 700,000, the polymer comes to resist flowing even under heating: as a result, high temperature and high pressure become necessary in making a preliminary molded body by melt molding, the heat produced by high shearing stress and friction force at the time of working causes a significant drop in molecular weight of the polymer, and the molecular weight of the finally obtained material becomes rather lower than that in the case of using one in which the molecular weight is 700,000 or lower, or equivalently, the strength becomes lower than expected value.

As to a polymer having a low initial viscosity-average molecular weight of from 150,000 to 200,000, it is possible to fill the polymer with a surface-bioactive bioceramic powder in a relatively large amount of 30 wt % to 60 wt %. However, because a further decrease in molecular weight of the polymer occurs after melt molding and thereby the molded body becomes apt to break when yields to an external force such as bending deformation (yield breakage), it is preferred that the filling amount be adjusted to a low value of 10 wt % to 30 wt %, and besides, the deformation degree R mentioned hereafter be controlled to a relatively small value. On the other hand, it is relatively difficult to melt-mold a polymer having a high viscosity-average molecular weight of from 550,000 to 700,000, and it is furthermore difficult to fill the polymer with a surface-bioactive bioceramic powder in a large amount of 40 wt % to 60 wt % and carry out melt-molding of the resultant polymer. Accordingly, the filling amount of a surface-bioactive bioceramic powder has to be adjusted to be below 20 wt % and the deformation degree R should be controlled to a small value. Briefly speaking, when the initial viscosity-average molecular weight is in a range of from 200,000 to 550,000, it is possible to choose the filling amount and the deformation degree R from relatively wide ranges, and besides, the strength is retained in a living body for an appropriate period and moderate degradation and absorption speeds are also attained.

When the filler is filled in a large amount, the resultant mixture becomes poor in flowability. Accordingly, for the purpose of allowing easy molding by lowering of melt viscosity, it is all right to add a low molecular-weight polymer having a viscosity-average molecular weight of 100,000 or lower or in some cases 10,000 or lower, as a lubricant in such a small amount as to exert no influence on physical properties of materials. When monomers remain in a large quantity in a polymer used, the polymer suffers a drop in molecular weight in the process of working, and in-vivo degradation thereof progresses fast. Accordingly, it is preferred that the addition amount of such a polymer be controlled to below 0.5 wt % or so. In a highly filling case where the filler content is higher than 40 wt %, for the purpose of increasing binding force at the interface between both ingredients, it is all right to use a filler having undergone surface treatment with a soft in-vivo absorbable polymer or a complex constituted of optical isomers of polylactide, namely poly-L-lactide and poly-D-lactide. Thus, through the molecular (crystal) alignment operation by subsequent press-fit filling into a mold, a high-strength aligned compact can be obtained without attended by a substantial drop in molecular weight.

### (e) Crystallinity

With consideration given to a balance between two factors required of an aligned compact formed through the press-fit filling, specifically a balance between high mechanical strength and moderate hydrolysis speed requirements, the aligned compact is required to choose its crystallinity from a range of 10% to 70%, preferably 20% to 50%. When its crystallinity exceeds 70%, the aligned compact is high in apparent rigidity, but poor in tenacity: as a result, it becomes brittle. In addition, such high crystallinity is undesirable because the degradation speed becomes lower than required and a long period of time is required for such highly crystallized one to be absorbed and disappear into a living body. Contrary to the above, for the case of low crystallinity below 10%, enhancement of strength by crystalline alignment cannot be desired. Under these circumstances, in consideration of mechanical strength and speed of disappearance by degradation and absorption or weak irritation to a living body, the appropriate crystallinity is from 10% to 70%, preferably 20% to 50%. Even when the crystallinity is as low as 10% to 20%, the strength is enhanced by filler effect as compared with the non-filling case. In addition, even the crystallinity as high as 50% to 70% little brings about disadvantageous action on degradation and absorption in a living body because such crystallinity induces formation of crystallites in the process of plastic deformation by pressurization. In other words, it is preferred that the crystallinity of the aligned compact be from 10% to 70%.

### (f) Density

The thus three-dimensionally aligned compact becomes high in density as compared with e.g. a molded body having undergone drawing alignment. Depending on the deformation degree, the density of an aligned compact having a mixing amount of a surface-bioactive bioceramic on the order of 20% is from 1.4 g/cm³ to 1.5 g/cm³, that of an aligned compact having the mixing amount on the order of 30% is from 1.5 g/cm³ to 1.6 g/cm³, that of an aligned compact having the mixing amount on the order of 40% is from 1.6 g/cm³ to 1.7 g/cm³, and that of an aligned compact having the mixing amount on the order of 50% is from 1.7 g/cm³ to 1.8 g/cm³. Such high density is an index indicating compactness and one of important factors supporting high strength.

### (g) Crystal Form

Because this material has undergone pressure alignment by press-fit filling during the making process, crystals (molecular chains) in the resultant compact align essentially parallel to a plurality of reference axes. In general, the larger the reference axes in number, the less anisotropic the compact in strength. Thus, in contrast to materials having directional properties, occurrence of breakage of the compact by relatively weak force from some direction becomes low in frequency.

### (B) Manufacturing Method

The manufacturing method is characterized basically in that (a) a mixture is prepared in advance by mixing and dispersing homogeneously in a substantial sense a surface-bioactive bioceramic powder into an in-vivo degradable and absorbable crystalline thermoplastic polymer, (b) next the mixture is made into a preliminary molded body (e.g. billet) by melt molding, and (c) then the preliminary molded body is made into an aligned compact by being filled by press-fit filling into the cavity of a closed mold having a narrow space between shaping dies the lower end of which is closed in a substantial sense (in the case of compression alignment) or into a narrow space of a mold the cross section of which is partially or wholly smaller in either a thickness or a width than that of the preliminary molded body or into the cavity of a mold whose space is made smaller in volume than the space to accommodate the preliminary molded body (in the case of forging alignment). Put another way, the manufacturing method comprises preparing in advance a mixture from an in-vivo degradable and absorbable, crystalline thermoplastic polymer and a surface-bioactive bioceramic powder by dispersing the powder into the polymer homogeneously in a substantial sense, then making the mixture into a preliminary molded body by melt-molding, and further making the preliminary molded body into an aligned compact through the plastic deformation by cold press-fit filling into the cavity of a closed mold.

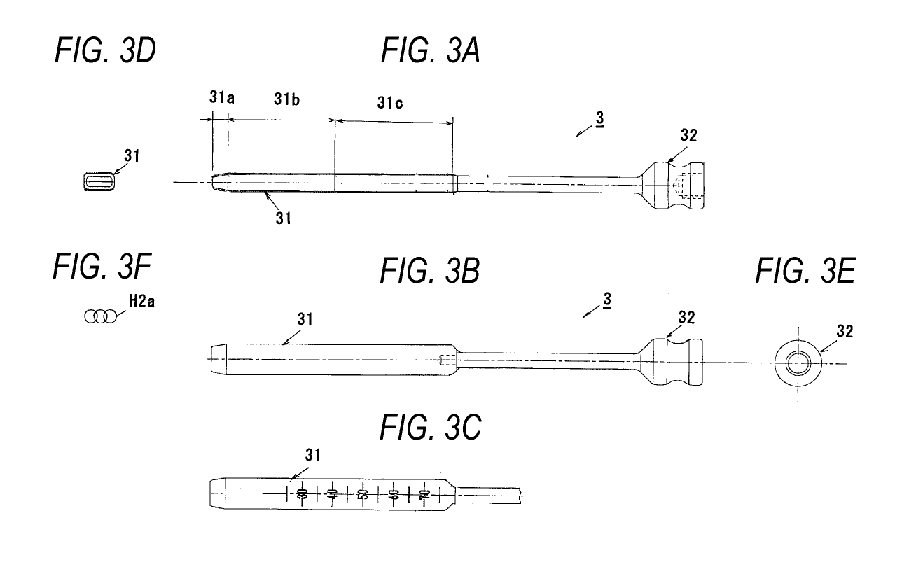

By the way, in order to form a hole H2 for fitting an in-vivo bone B2 rectangular in traverse-sectional profile, it is intended to use a bone hole forming tool 3 (dilator) as shown in FIGS. 3A-3F.

The bone hole forming tool 3 is inserted into a living body and forms the fitting hole H2 by shaving. In order to do so, this tool is configured to have the surface having received shot blast treatment and include a square tube-shaped front-end part 31 with a scale and a calibration line drawn by laser marking and an operating part 32 capable of delivering a blow or the like to a base-end side situated outside a living body.

And the front-end part 31 includes a taper section 31a steep on the tip side, a gentle taper section 31b and a parallel section 31c, and the traverse-sectional profile thereof has the same dimension as or a little larger dimension, specifically a dimension greater by 0 to 1.0 mm, preferably 0 to about 0.5 mm, than that of the front-end part 10a of the ligament fixture 1. By the use of such a bone hole forming tool, the fitting hole H2 rectangular in traverse-sectional profile and having a traverse-sectional profile smaller than that of the main body 10b of the ligament fixture 1 is formed in the in-vivo bone B2.

In addition, by forming the gentle taper section 31b so as to have a long length, it becomes possible to form in an in-vivo bone B2 a fitting hole H2 having a traverse-sectional profile the size of which decreases gradually in the depth direction.

In forming a fitting hole H2 having a rectangular traverse-sectional profile in an in-vivo bone, as shown in FIG. 3F, a lower hole H2a with a range of many holes (3 holes in the figure) is formed first in an in-vivo bone B2 by means of a drill, and then the lower hole H2a is shaven by means of the bone hole forming tool 3 so as to become enlarged and form a predetermined fitting hole H2 rectangular in traverse-sectional profile.

In the foregoing ways, it becomes possible to insert and fix the ligament fixture 1 into the fitting hole H2 which has been formed in an in-vivo bone B2 so as to have a cross-sectional profile invariant in the depth direction or so as to have a cross-sectional profile reduced gradually in the depth direction. Thus, in response to properties of the in-vivo bone B2 intended for application, the insertion and fixation conditions of the ligament fixture 1 may be adjusted by making use of the shape of the fitting hole H2.

When it is intended to implant a ligament by the use of the ligament fixture 1, as shown in FIG. 4A, holes H1 and H2 are formed in the in-vivo bones (articular bones) B1 and B2, respectively, which are situated on opposite sides of a place for fitting the ligament M, by means of a drill and the bone hole forming tool 3. By drawing a connecting member W2 through a hole (a through hole 15) formed in each of the ligament fixture 1 and one bone piece Bo in a condition that the ligament fixture 1 and the bone piece Bo ranging to the ligament M are made to closely adjoin to each other, the ligament fixture 1 and the bone piece Bo are connected and fixed together. To the other bone piece Bo ranging to the ligament M is connected a string-shaped connecting member W1 as a guiding suture. The thus connected thing is drawn through the hole H2 and the hole H1 in order of mention so as to head to the hole H1 formed in the in-vivo bone B1 from the hole H2 formed in the in-vivo bone B2 in a state that the ligament fixture 1 is situated at the rear end. And the connected thing is pulled into the holes so that the ligament M lies between the in-vivo bone B1 and the in-vivo bone B2 and the bone pieces Bo ranging to the ligament M are situated in the holes H1 and H2 formed in the in-vivo bones B1 and B2, respectively.

And by delivering a blow or the like to the rear end of the ligament fixture 1 through the medium of the operation tool 2, the ligament fixture 1 is inserted and fixed into the fitting hole H2 formed in the in-vivo bone B2, and through the medium of this ligament fixture 1, one bone piece Bo ranging to the ligament M is engaged in the mouth of the hole H2 formed in the in-vivo bone B2, and further the ligament M is kept in a tension-applied state so as not to slack by pulling the string-shaped connecting member W1 as a guiding suture. In this situation, by screwing an interference screw S made from an in-vivo degradable and absorbable material into the space between the other bone piece Bo placed in the hole H1 formed in the in-vivo bone B1 and the hole H1, and thereby the bone piece Bo is engaged in a state of being sandwiched between the circumferential wall of the hole H1 and the peripheral face of the interference screws, and thereby the implantation of the ligament M is completed.

The ligament fixture 1 and the bone piece Bo are linked and fixed together without clearance in the foregoing way, and thereby not only a strong linkage is formed with stability, but also the cavity in the hole H2 is reduced, which brings an advantage that the clearance in the hole H2 can be healed early with a newly formed bone.

In this case, as shown in FIG. 4B, it is preferred that the whole of the ligament fixture 1 be inserted and fixed so as to be buried in the fitting hole H2 formed in the in-vivo bone B2.

By being buried in such a condition, the ligament fixture 1 can be prevented from causing a detrimental effect, such as inflammation during the convalescence, by protruding through the surface of the in-vivo bone B2.

And because of containing a bioceramic having bioactivity, the ligament fixture 1 accelerates the formation of a nascent bone: as a result, bone formation or replacement is performed, and the ligament M is fixed firmly to the in-vivo bones B1 and B2 through the medium of the bone pieces Bo placed at both its ends, thereby completing the reconstruction.

In addition, in the present manipulation, a ligament is extracted from a living body as a bone piece Bo is attached to either end of the ligament and put to use, and hence there may be cases where the ligament is broken by physical activities after a lapse of a certain period of time from the surgical operation to result in reoperation becoming necessary. In even such cases, because of the use of an in-vivo degradable and absorbable material in the ligament fixture 1, it is possible to form the similar hole H2 by means of a drill or the like at the site of the last-time operation. This possibility makes an excellent characteristic. In the traditional manipulation using a fixture made from a metal, a hole cannot be made at the site of the last-time operation, and the position of the hole is therefore changed and reoperation is carried out. Thus it becomes impossible to accomplish the objective of reconstructing a ligament at its original site in a living body. In comparison with such a situation, the foregoing possibility can be regarded as an excellent advantage of the present manipulation.

### EXAMPLES

In the next place, more specific shapes (specifications) and strength test results of samples of the ligament fixture 1 are illustrated.

The shapes (specifications) of samples of the ligament fixture 1 used for strength tests are as follows.

Ligament fixture (A)

Length L: 15 mm

Rear-end width Wa: 10.5 mm

Front-end width Wb: 9.5 mm

Rear-end thickness Ta: 7.0 mm

Front-end thickness Tb: 6.0 mm

Protrusion height H: 1 mm

Through hole diameter d: φ3 mm

Groove width Wg: 3 mm

Ligament fixture (B)

Length L: 15 mm

Rear-end width Wa: 10.5 mm

Front-end width Wb: 9.5 mm

Rear-end thickness Ta: 7.5 mm

Front-end thickness Tb: 6.5 mm

Protrusion height H: 1 mm

Through hole diameter d: φ3 mm

Groove width Wg: 3 mm

In each of the strength tests, after holes H1 and H2 were formed in femurs of a pig, respectively, by means of a drill and the bone hole forming tool 3, a metal stick was used instead of a ligament, and the metal stick and a ligament fixture 1 were connected and fixed together by means of a connecting member (suture). Then, the ligament fixture 1 engaged in the end part of the connecting member was received a blow via an operation tool 2, and thereby inserted and fixed into the fitting hole H2 formed in the in-vivo bone B2, and further the metal stick was drawn from the opposite side through the medium of a connecting member (suture). Under these circumstances, maximum load measurements were performed.

Results of strength tests are shown in Table 1.

TABLE 1

| | | | Dilator (thickness of | Maximum | Average |
|---|---|---|---|---|---|
| No. | Ligament fixture | Suture | parallel section (mm)) | load (N) | value (N) |
| 1 | (A) | High-strength | 6 | 761.1 | 785.8 ± 22.9 |
| 2 | Rear-end | suture | | 781.9 | |
| 3 | thickness: 7.0 mm | (one piece: double) | | 782.1 | |
| 4 | Front-end | | | 823.6 | |
| 5 | thickness: 6.0 mm | | | 780.1 | |
| 6 | (B) | | 6.5 | 799.7 | 808.2 ± 42.1 |
| 7 | Rear-end | | | 848.6 | |
| 8 | thickness: 7.5 mm | | | 831.8 | |
| 9 | Front-end | | | 752.9 | |
| | thickness: 6.5 mm | | | | |

In every sample shown in Table 1, its ligament fixture 1 caused neither slip nor breakage, and the maximum load thereon became the load under which the connecting member (suture) came to break. Thus, it was verified experimentally that the maximum load applicable to every sample far exceed the strength required for practical use.

From these data, it has been ascertained that each ligament fixture 1 can maintain a good fixing condition until the time comes when the connecting member (suture) will break.

In addition, each ligament fixture 1 was inserted and fixed into the fitting hole H2 formed in the in-vivo bone B2 by receiving a blow via the operation tool 2, but neither fracture nor like damage was caused thereby.

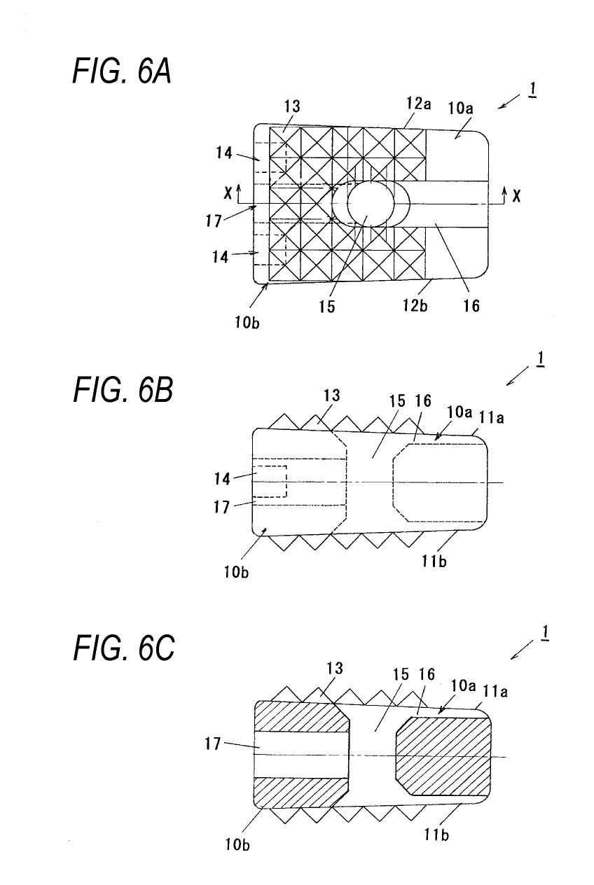

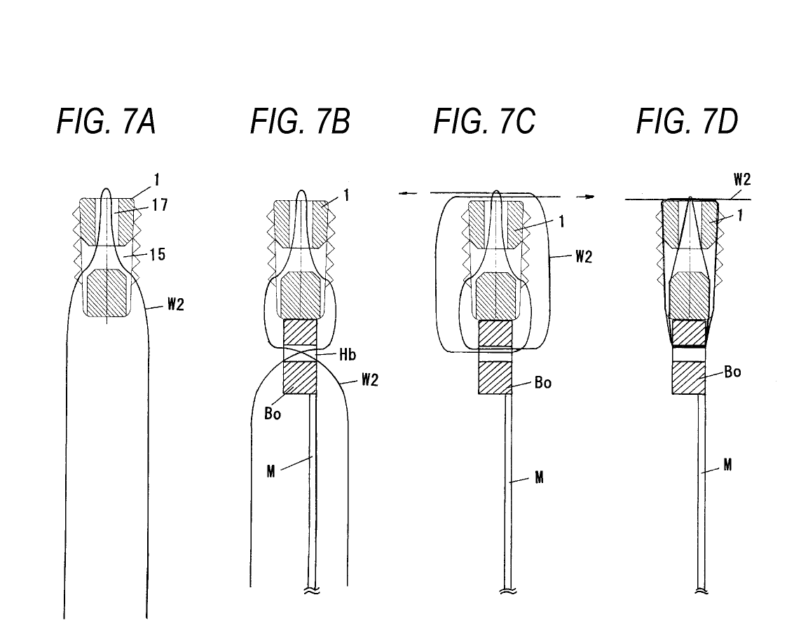

By the way, although each of the ligament fixtures 1 shown in FIGS. 1A-1B and FIGS. 2A-2B was configured to form in its intermediate part the through hole 15 for engagement of the connecting member W2 to be connected to a ligament M, it is also possible that, as shown in FIGS. 6A-6C and FIGS. 7A-7D, a communication hole 17 ranging from the rear-end face of the ligament fixture 1 to the through hole 15 formed in the intermediate part of the ligament fixture 1 is further formed, the intermediate part of the string-shaped connecting member W2 to be connected to a ligament M is situated in the form of a loop at the opening part of the communication hole 17 on the rear-end face side of the ligament fixture 1 and both the end parts of the connecting member W2 are arranged so as to be pulled out from both openings of the through hole 15, respectively.

Herein, connection between the ligament fixture 1 and the ligament M through the use of the connecting member W2 is performed in the following manner. Both ends of the connecting member W2 arranged so as to be pulled out from both openings of the through hole 15 respectively (FIG. 7A) are inserted from both directions into a hole Hb formed in the bone piece Bo ranging to the ligament M (FIG. 7B), and then inserted from both directions into the loop-shaped intermediate part situated at the opening part on the rear-end face side of the ligament fixture 1 (FIG. 7C) and tighten up so as not to allow any slack in the connecting member W2 (FIG. 7D).

In such a situation, when the ligament M implanted in a living body is subjected to tension, the connecting member W2 that connects between the ligament fixture 1 fitted into the in-vivo bone B2 and the bone piece Bo is placed under tension. In this section, the connecting member W2 has four pieces, two among these pieces form the loop, and both end parts of the connecting member W2 are inserted into the loop and secured. Herein, although forces act in directions that the loop forming part of the connecting member W2 is pulled into the communication hole 17, it is impossible for both the end parts of the connecting member W2, which have passed through the loop, to enter into the communication hole 17 because the communication hole 17 was made so as to have a diameter allowing passage of two pieces, not three pieces, of the connecting member W2, and thereby the connecting member W2 is prevented from being untied (Diagrams in FIGS. 7A-7D are schematically drawn, wherein the diameter of the communication hole 17 and the thickness of the connecting member W2 are chosen so as to satisfy such a relation). More specifically, the higher the tension applied to the connecting member W2 that connects between the ligament fixture 1 and the bone piece Bo, the more tightly the loop of the connecting member W2 is tied, and thereby both end parts of the connecting member W2, which have passed through the loop, are protected from becoming untied.

Thus, it becomes possible to connect between the ligament fixture 1 and the ligament M through the use of the connecting member W2 with ease and certainty.

Herein, it is preferred that the front-end face at which the ligament fixture 1 abuts against the bone piece Bo ranging to the ligament M be formed in a flat face so as to come into surface contact with the bone piece Bo when the ligament fixture 1 is connected to the ligament M by means of the connecting member W2.

When the front-end face of the ligament fixture 1 is formed so as to be flat in the thickness equivalent to that of the bone piece Bo, it becomes possible that the bone piece Bo and the ligament fixture 1 are tightly connected together with stability by means of the connecting member W2.

When the ligament fixture 1 and the ligament M are brought into surface contact and no clearance is left between them, bone conductivity (which means that, when a medical material is buried into an in-vivo bone, a new bone is formed along the material surface, and the new bone and the material are bound together and integrated into one) owing to bioceramics incorporated into the ligament fixture 1 comes into play, and thereby it becomes possible to accelerate bone formation between the ligament fixture 1, the ligament M and the hole H2, which is formed in the in-vivo bone B2 and communicates with the former two, and hence expedite repair.

Additionally, in this case, it is arranged that the linkage site 14 for connecting an operation tool 2 with which are performed operations for fitting the ligament fixture 1 in the human body is formed on either side of the opening part of the communication hole 17 and, as the operation tool, one which has a bifurcated tip is used.

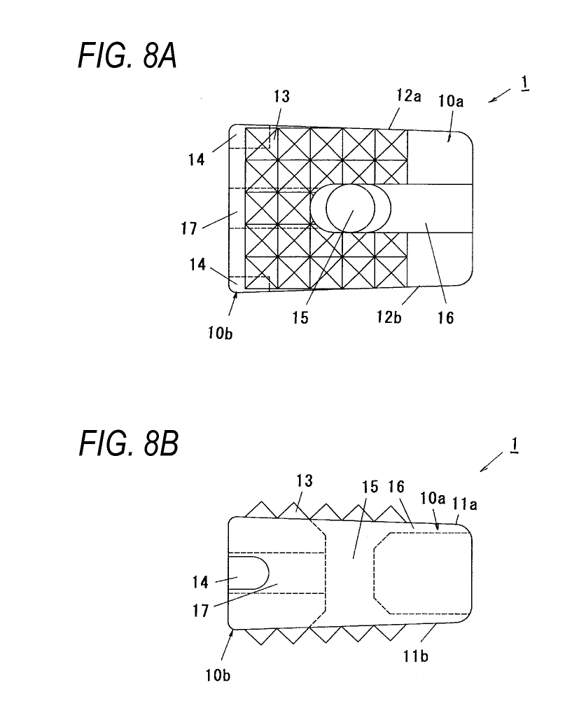

Alternatively, as shown in FIGS. 8A-8B, it is also possible for the linkage site 14 to be formed on either side of the ligament fixture 1 and for the operations to be performed by fastening both sides of the ligament fixture 1 with an operation tool having a bifurcated tip.

In the foregoing, the ligament fixture according to an aspect of the invention has been illustrated by reference to embodiments thereof. However, the invention should not be construed as being limited to the constitution of each of the above-mentioned embodiments, and various changes and modifications can be made appropriately as to the constitution without departing from the spirit and scope of the invention.

Because of having properties of ensuring high certainty and operability in fixing a connecting member connected to a ligament, and besides allowing not only easy adaption to the ligament having a traverse-sectional profile long in its lateral direction but also reduction in burdens on patients, the ligament fixture can be used suitably for the purpose of implanting a ligament (or tendon) into the knee joint concerned in reconstructive surgery of an anterior cruciate ligament (ACL) torn.

What is claimed is:

1. A ligament fixture to be inserted and fixed into a fitting hole formed in an in-vivo bone, comprising an in-vivo degradable and absorbable material which is a polymer-bioceramic composite material,
the ligament fixture having at least one pair of opposed surfaces which are inclined faces gradually getting closer to each other toward to a front-end side of the ligament fixture and which form a wedge shape having a resistance imparting parts on each of the inclined faces, and
the ligament fixture comprising a front-end part having a cross-sectional profile smaller than a cross-sectional profile of the fitting hole, and a main body part continuing from the front-end part, the main body part comprising a section having a cross-sectional profile larger than the cross-sectional profile of the fitting hole.

2. The ligament fixture according to claim 1, having the other pair of opposite surfaces which are inclined faces gradually getting closer to each other toward the front-end side of the ligament fixture.

3. The ligament fixture according to claim 1, wherein the resistance imparting parts are provided on the section having the cross-sectional profile larger than the cross-sectional profile of the fitting hole.

4. The ligament fixture according to claim 1, wherein the resistance imparting parts are formed so as to protrude from the inclined faces.

5. The ligament fixture according to claim 1, wherein the resistance imparting parts are formed with recesses provided in the inclined faces.

6. The ligament fixture according to claim 1, further comprising a rear-end part having a linkage site for connecting an operation tool.

7. The ligament fixture according to claim 1, further comprising an intermediate part having a through hole for engaging therein a connecting member to be connected to a ligament, and having a groove formed from the through hole toward the front-end part for placing the connecting member.

8. The ligament fixture according to claim 7, wherein the intermediate part has a communicating hole ranging from a rear-end face of the ligament fixture to the through hole in the intermediate part, and
an intermediate part of the connecting member to be connected to the ligament is positioned in a form of a loop at an opening part of the communicating hole on a rear-end face of the ligament fixture, and both end parts of the connecting member is disposed so as to be pulled out of both openings of the through hole, respectively.

9. The ligament fixture according to claim 1, which is made from the composite material having undergone reinforced molding.

10. The ligament fixture according to claim 1, which is configured to be inserted and fixed into the fitting hole formed in an in-vivo bone in a condition of being wholly buried in the fitting hole.

11. The ligament fixture according to claim 1, which is configured to be inserted and fixed into the fitting hole formed in an in-vivo bone by means of a bone hole forming tool so as to have a cross-sectional profile invariant in a depth direction.

12. The ligament fixture according to claim 1, which is configured to be inserted and fixed into a fitting hole formed in an in-vivo bone by means of a bone hole forming tool so as to have a cross-sectional profile gradually reducing in a depth direction.
